(19) United States
(12) Patent Application Publication
(10) Pub. No.: US 2023/0157881 A1
(43) Pub. Date: May 25, 2023

(54) IMAGING AND TREATING A VITREOUS FLOATER IN AN EYE

(71) Applicant: Alcon Inc., Fribourg (CH)

(72) Inventors: John Park, Irvine, CA (US); Edward A. DeHoog, Long Beach, CA (US)

(21) Appl. No.: 18/049,986

(22) Filed: Oct. 26, 2022

Related U.S. Application Data
(60) Provisional application No. 63/281,498, filed on Nov. 19, 2021.

(51) Int. Cl. A61F 9/008

(52) U.S. Cl. CPC A61F 9/008; A61F 2009/00851; A61F 2009/00897

(57) ABSTRACT

In certain embodiments, an ophthalmic laser surgical system for imaging and treating a target in an eye includes an imaging system. The imaging system includes a scanning laser ophthalmoscope (SLO) device and an optical coherence tomography (OCT) device. The SLO device generates SLO images, and the OCT device generates OCT images. The SLO device and the OCT device share a scanning system and a light detector. The scanning system scans SLO and OCT imaging beams within the eye. The light detector detects the SLO and OCT imaging beams reflected from the eye and generates SLO and OCT signals in response to detecting the imaging beams.

### TECHNICAL FIELD

The present disclosure relates generally to ophthalmic surgical systems, and more particularly to imaging and treating a floater within an eye.

### BACKGROUND

Vitreous eye floaters are microscopic collagen fibers that clump together and disturb the vision of the patient. Laser vitreolysis treats this condition by directing a laser beam into the vitreous towards a floater. The laser beam fragments and removes the floater to improve vision.

### BRIEF SUMMARY

In certain embodiments, an ophthalmic laser surgical system for imaging and treating a target in an eye includes an imaging system, a treatment system, and a computer. The imaging system directs imaging beams into the eye to generate images of the target within the eye. The imaging beams include a scanning laser ophthalmoscope (SLO) imaging beam and an optical coherence tomography (OCT) imaging beam. The imaging system includes an SLO device and an OCT device. The SLO device includes a scanning system, a light detector, and an SLO detector. The scanning system scans the SLO imaging beam within the eye. The light detector generates an SLO signal in response to detecting the SLO imaging beam reflected from the eye. The SLO detector generates SLO images from the SLO signal. The OCT device includes the scanning system, the light detector, and an OCT detector. The scanning system scans the OCT imaging beam within the eye. The light detector generates an OCT signal in response to detecting the OCT imaging beam reflected from the eye. The OCT detector generates OCT images from the OCT signal. The treatment system includes a laser device that directs a laser beam towards the target. The computer instructs the imaging system to generate the images and the laser device to direct the laser beam towards the target.

Embodiments may include none, one, some, or all of the following features:
The ophthalmic laser surgical system includes an imaging beam source that generates the imaging beams comprising the SLO imaging beam and the OCT imaging beam.
The computer instructs: the OCT device to scan the OCT imaging beam in a z-direction relative to the z-axis to generate an A-scan within the eye; and the SLO device to scan the SLO imaging beam in an xy-direction relative to the xy-planes to generate two-dimensional (2D) enface images.
The scanning system includes: an xy-scanner that scans an imaging beam in an xy-direction relative to an xy-plane within the eye; and a z-scanner that scans the imaging beam in a z-direction relative to the z-axis within the eye. The xy-scanner may: direct the imaging beams along an imaging beam path towards an xy-location of the target; and direct the laser beam along a laser beam path aligned with the imaging beam path towards the xy-location of the target. The z-scanner may: direct the imaging beams along an imaging beam path towards a z-location of the target; and direct the laser beam along a laser beam path aligned with the imaging beam path towards the z-location of the target. The z-scanner may include a corner cube moving mirror (CCMM) that moves to adjust a path length to create a coherence gate for maximum signal.
The light detector includes: a high-frequency filter that provides the SLO signal; and a low-frequency filter that provides the OCT signal.
The OCT detector includes a fringe counter that counts interference fringes.
The OCT device measures a z-location of the target relative to the z-axis.
The laser device receives z-location of the target from the imaging system and direct the laser beam towards the z-location of the target.
The computer: determines a radiant exposure at a retina of the eye resulting from the laser beam directed to the z-location of the target; and determines whether the radiant exposure is less than a maximum radiant exposure.
The SLO device generates two-dimensional (2D) enface images, each enface image located in a different xy-plane. The computer combines the plurality of 2D enface images to yield three-dimensional (3D) images. The computer may output the 3D images via a display.

In certain embodiments, a method for imaging and treating a target in an eye comprises directing, by an imaging system, imaging beams into the eye to generate images of the target within the eye. The imaging beams comprise a scanning laser ophthalmoscope (SLO) imaging beam and an optical coherence tomography (OCT) imaging beam. The eye has an eye axis that defines a z-axis, which in turn defines xy-planes orthogonal to the z-axis. The images of the target are generated by: scanning, by a scanning system of an SLO device of the imaging system, the SLO imaging beam within the eye; generating, by a light detector of the SLO device, an SLO signal in response to detecting the SLO imaging beam reflected from the eye; generating, by an SLO detector of the SLO device, a plurality of SLO images from the SLO signal; scanning, by the scanning system of an OCT device of the imaging system, the OCT imaging beam within the eye; generating, by the light detector of the OCT device, an OCT signal in response to detecting the OCT imaging beam reflected from the eye; and generating, by an OCT detector of the OCT device, a plurality of OCT images from the OCT signal. A laser beam is directed by a laser device of a treatment system towards the target within the eye. The imaging system is instructed by a computer to generate the images, and the laser device is instructed by the computer to direct the laser beam towards the target.

Embodiments may include none, one, some, or all of the following features:
The OCT device is instructed by the computer to scan the OCT imaging beam in a z-direction relative to the z-axis to generate an A-scan within the eye. The SLO device is instructed by the computer to scan the SLO imaging beam in an xy-direction relative to the xy-planes to generate two-dimensional (2D) enface images.
An imaging beam is scanned by an xy-scanner of the scanning system in an xy-direction relative to an xy-plane within the eye. The imaging beam is scanned by a z-scanner of the scanning system in a z-direction relative to the z-axis within the eye.
The imaging beams are directed by the xy-scanner along an imaging beam path towards an xy-location of the target. The laser beam is directed by the xy-scanner along a laser beam path aligned with the imaging beam path towards the xy-location of the target.
The imaging beams are directed by the z-scanner along an imaging beam path towards a z-location of the target. The laser beam is directed by the z-scanner along a laser beam path aligned with the imaging beam path towards the z-location of the target.
The z-scanner comprises a corner cube moving mirror (CCMM) that moves to adjust a path length to create a coherence gate for maximum signal.

### BRIEF DESCRIPTION OF THE DRAWINGS

FIG. 1 illustrates an example of an ophthalmic laser surgical system for imaging and treating a target in an eye, according to certain embodiments;

FIG. 2 illustrates examples of SLO 2D enface images that may be generated by the SLO device of FIG. 1, according to certain embodiments;

FIG. 3 illustrates an example of a corner cube moving mirror CCMM and mirror of the z-scanner of FIG. 1, according to certain embodiments; and

FIG. 4 illustrates an example of a method for imaging and fragmenting a target in an eye, which may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Laser vitreolysis uses a laser beam to fragment and remove eye floaters. Known systems for performing laser vitreolysis, however, fail to treat floaters effectively and efficiently. For example, known systems often use illumination that generates significant retinal reflections that degrade imaging of floaters. Moreover, the systems typically use a dual beam technique that cannot provide precise depth measurements.

Accordingly, the surgical systems described herein have a scanning laser ophthalmoscope (SLO) device that generates two-dimensional (2D) and three-dimensional (3D) images that provide clearer images of floaters. In addition, the systems include an optical coherence tomography (OCT) device that provides more precise depth measurements of the floaters. Moreover, the treatment and imaging systems of the surgical systems share components that allow for co-registration of treatment and imaging beams, as well as reduce manufacturing costs.

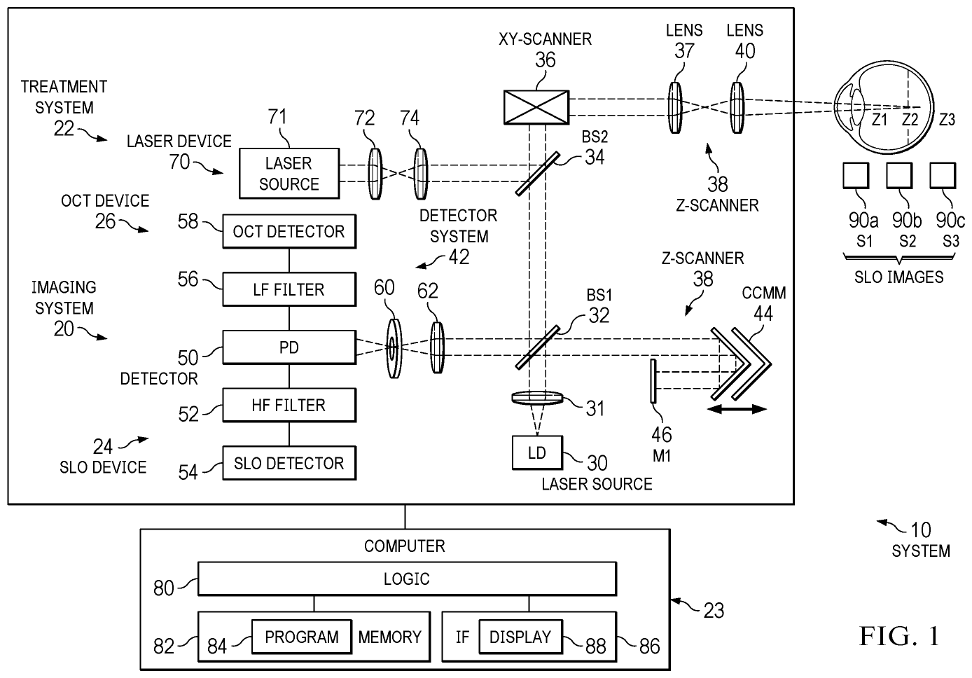

FIG. 1 illustrates an example of an ophthalmic laser surgical system 10 for imaging and treating a target in an eye, according to certain embodiments. In the example, the target may be a vitreous floater. The eye has an eye axis (e.g., visual or optical axis), which defines a z-axis. Z-locations and the z-direction are relative to the z-axis. The z-axis defines xy-planes orthogonal to the z-axis. Xy-locations and xy-directions are relative to an xy-plane.

As an overview of the illustrated example, system 10 includes an imaging and measuring system 20, a treatment system 22, and a computer 23, coupled as shown. Imaging system 20 includes an SLO device 24 and an OCT device 26, coupled as shown. SLO device 24 and OCT device 26 share certain components of system 10, including an imaging laser source 30 (e.g., laser diode LD), a lens 31, a beamsplitter BS1 32, a beamsplitter BS2 34, an xy-scanner 36, a lens 37 (which operates with a z-scanner 38), z-scanner 38, an objective lens 40, and a detector system 42. Z-scanner 38 includes a corner cube moving mirror CCMM 44, a mirror M1 46, and lens L1 37, coupled as shown.

Continuing with the overview, detector system 42 includes a detector 50 (e.g., photodiode PD), a high-frequency (HF) filter 52, an SLO detector 54, a low-frequency (LF) filter 56, an OCT detector 58, a small aperture such as pinhole 60, and a lens 62, coupled as shown. Regarding detector system 42, SLO device 24 utilizes HF filter 52 and SLO detector 54, and OCT device 26 utilizes LF filter 56 and OCT detector 58. Treatment system 22 includes a laser device 70, which includes a treatment laser source 71 and lenses 72, 74, coupled as shown. Treatment system 22 includes and shares beamsplitter BS2 34, xy-scanner 36, lens 37, and objective lens 40 with imaging system 20, coupled as shown. Computer includes logic 80, a memory 82 (which stores a program 84), and interface 86 (which includes a display 88), coupled as shown.

As an example of operation, laser source 30 generates imaging beams, including SLO and OCT imaging beams. Imaging system 20 directs the imaging beams into the eye to generate images of the target within the eye, which reflects the imaging beams. At the SLO device 24, a scanning system (comprising xy-system 36 and z-scanner 38) scans the focus of the SLO imaging beam within the eye. Light detector 50 generates an SLO signal in response to sensing the SLO imaging beam reflected from the eye. SLO detector 54 generates SLO images 90 (90a, 90b, 90c) from the SLO signal.

Continuing with the example of operation, at OCT device 26, the scanning system scans the focus of the OCT imaging beam within the eye. Light detector 50 generates an OCT signal in response to sensing the OCT imaging beam reflected from the eye. OCT detector 58 generates OCT images from the OCT signal. Laser device 70 directs a laser beam along a laser beam path towards the target within the eye. Computer 23 instructs the imaging system to generate the images and the laser device to direct the laser beam along the laser beam path towards the target.

Turning to the components, imaging system 20 and treatment system 22 direct light beams towards the interior of the eye. Imaging system 20 generates images of the eye interior, including images of the target (e.g., a floater) within the eye. In imaging system 20, SLO device 24 generates SLO images, and OCT device 26 generates OCT images. Imaging laser source 30 generates imaging beams, including SLO and OCT imaging beams. Imaging laser source 30 may comprise any suitable laser source that generates laser beams of any suitable wavelength. For example, laser source 30 may generate OCT imaging beams in the near-infrared (NIR) range, such as 750 to 1400 nanometers (nm). In general, the central wavelength of OCT imaging beams is selected to achieve maximal penetration depth into the tissue under examination. For ophthalmic systems, the wavelength is typically approximately 850 nm or approximately 1050 nm to allow light penetration through the retinal pigment epithelium (REP) to enable imaging of the choroid. Laser source 30 may generate SLO imaging beams in the visible to NIR range, such as 380 to 910 nm. Lens 31 collimates the imaging beams.

Beamsplitter BS1 32 splits the imaging beams to yield a sample beam for imaging the eye and a reference beam for the reference arm of OCT device 26. Generally, the imaging beam is split such that the sample beam has greater intensity than that of the reference beam, e.g., the sample beam intensity is 70% to 90% such as 80% of the intensity of the imaging beam. Beamsplitter BS1 32 also directs imaging beams reflected from the eye towards detector system 42. Beamsplitter BS1 32 may comprise any suitable beamsplitter, e.g., a metallic and/or dielectric thin film applied to an optical substrate, a polarization beamsplitter, or a pellicule that allows partial transmission and/or reflection of the incident beam.

Treatment system 22 includes a laser device 70 that directs a treatment laser beam into the eye to fragment the target. Treatment laser source 30 generates treatment beams, and may comprise any suitable laser source that generates laser beams of any suitable wavelength, e.g., 1030 to 1065 nanometers (nm). Lenses 72, 74 collimate the treatment beams. Lenses 72 and 74 may be an optical relay that focuses the laser beam at the target while maintaining image focus.

Beamsplitter BS2 34 transmits imaging and treatment beams to xy-scanner 36. Beamsplitter BS2 34 also directs imaging beams reflected from the eye towards beamsplitter BS1 32. Beamsplitter BS2 34 may comprise any suitable beamsplitter, e.g., a dichroic mirror, a polarization beamsplitter, or a partially reflective metallic or dielectric thin film applied to an optical substrate. A dichroic mirror allows for different wavelength to be combined along the same optical path to couple the imaging and treatment beam paths. From beamsplitter BS2 34 to the eye, the imaging and treatment beam paths are aligned.

A scanning system scans laser beams in the x-, y-, and z-directions within the eye. In the example, the scanning system includes xy-scanner 36 that scans beams in xy-directions and z-scanner 38 that scans in the z-direction. The imaging and treatment beam paths are aligned, so a scanner can direct imaging and treatment beams to the same location. For example, xy-scanner 36 directs imaging and treatment beams along the imaging and treatment beam paths towards the xy-location of the target. As another example, z-scanner 38 directs imaging and treatment beams along the imaging and treatment beam paths towards a z-location of the target.

Xy-scanner 36 scans treatment and imaging beams transversely in xy-directions. Examples of scanners include a galvo scanner (e.g., a pair of galvanometrically-actuated scanner mirrors that can be tilted about mutually perpendicular axes), an electro-optical scanner (e.g., an electro-optical crystal scanner) that can electro-optically steer the beam, or an acousto-optical scanner (e.g., an acousto-optical crystal scanner) that can acousto-optically steer the beam. XY-scanner 36 may include an afocal relay lens system that allows for the compensation of patient refractive error. The afocal relay lens system may be used to image the scanner onto the pupil of the eye such that, while the optical beam is traversing the retina, the movement of the beam at the pupil of the eye is minimized to reduce or eliminate vignetting. The afocal relay lens system can be designed such that the scanner is conjugate to the pupil and movement of the lenses in the afocal relay (relative spacing between lenses 37 and 40) can accommodate for patient refractive error.

Z-scanner 38 includes corner cube moving mirror CCMM 44, mirror M1 46, and lens L1 37. CCMM 44 moves relative to beamsplitter BS1 32 and mirror M1 46 to adjust an imaging and/or treatment path length to create a coherence gate where the imaging and/or treatment beam paths are phase matched for maximum signal. CCMM 44 is described in more detail with reference to FIG. 3. Objective lens 40 focuses treatment and imaging beams within the eye.

The eye reflects the imaging beams. Detector system 42 receives imaging beams reflected from the eye and generates SLO and OCT images from the reflected beams. Lens 62 focuses the beams through a small aperture or pinhole 60, which rejects out-of-focus light from the sources other than the object of interest. Pinhole 60 is optically conjugate to the imaging plane Z2 in the eye and rejects reflected/backscattered light from any object not located at plane Z2. Detector 50 detects the reflected beams and generates a signal in response to the beams. Detector 50 may comprise, e.g., a photodiode. HF filter 52 provides an SLO signal to SLO detector 54, and LF filter 56 provides the OCT signal to OCT detector 58. OCT detector 58 may comprise any suitable device that can translate interference signals to an images, e.g., a fringe counter configured to count interference fringes.

SLO device 24 generates SLO images of the interior of the eye. In general, SLO device 24 can provide higher field of view (FOV) imaging, which may facilitate detection of moving floaters or other vitreous opacities during treatment. Moreover, SLO images enhance the contrast between a floater (or floater shadow) and the retina, allowing for easier detection of floaters.

In certain embodiments, SLO device 24 generates two-dimensional (2D) enface images, where each enface image is located in a different xy-plane. For example, a retinal enface image may be taken at or near the retina. The image may show floater shadows that can be used to assess the visual impact of floaters. As another example, a target 2D image may be taken at or near the target to show the floater. In certain embodiments, computer 23 combines the 2D enface images to yield three-dimensional (3D) images of the target within the eye. In the embodiments, computer 23 may output the images via display 88.

OCT device 26 generates OCT images of the interior of the eye and may be any suitable OCT device, e.g., a time domain OCT (TD-OCT) device. A TD-OCT can scan an entire eye at high speed with micron level precision, so can quickly measure the z-location (i.e., depth) of a floater within a few microns. In certain embodiments, OCT device 26 measures the z-location of the target, and laser device 70 receives the z-location of the target from the imaging system 20 and directs the laser beam towards the z-location of the target.

In certain embodiments, OCT device 26 sends a reference signal to the reference arm and a sample signal to the eye and detects the reflected signals. The reference path length is adjusted by moving CCMM 44. As the reference path length is adjusted, detector 50 detects alternating bright and dark signals, i.e., optical fringes, to generate an A-scan (z-direction scan). The optical fringes are analyzed to measure the z-locations of, e.g., the target (e.g., the floater), laser beam focus, and/or anatomical feature of the eye (e.g., lens and/or retina).

Computer 23 controls the operation of system 10, e.g., may control the operation of imaging system 20 and/or treatment system 22. In certain embodiments, computer 23 instructs OCT device 26 to scan an OCT imaging beam in a z-direction to generate an OCT A-scan, and instructs SLO device 24 to scan an SLO imaging beam in an xy-direction to generate two-dimensional (2D) enface images. The OCT and SLO imaging beams may scanned simultaneously. In certain embodiments, computer 23 performs image procession on an image to evaluate the visual impact of a floater. For example, the size of a floater's shadow indicates the size of the floater. As another example, the contrast of a floater's shadow relative to the retina indicates the density and/or thickness of the floater.

In certain embodiments, computer 23 determines a radiant exposure at the retina resulting from a laser beam directed to the z-location of the target, and determines whether the radiant exposure is greater or less than a maximum acceptable radiant exposure. In the embodiments, computer 23 calculates the radiant exposure Hₑ by determining a laser spot size of the laser beam on the retina and calculating the radiant exposure Hₑ according to the target-to-retina distance ΔZ and the laser spot size on the retina. For example, the laser spot diameter Φ may be calculated according to Φ=2*ΔZ*tan α, where α represents the known half angle of the cone of the laser beam. The radiant exposure Hₑ may be calculated according to Hₑ=4*E/Φ²*π=4*E/(2*ΔZ*tan α)²π, where E is the energy of the laser pulse. If the radiant exposure is greater than a maximum acceptable radiant exposure, computer 23 may notify the user, adjust the energy of the laser beam, and/or prevent the laser from firing to avoid overexposing the retina.

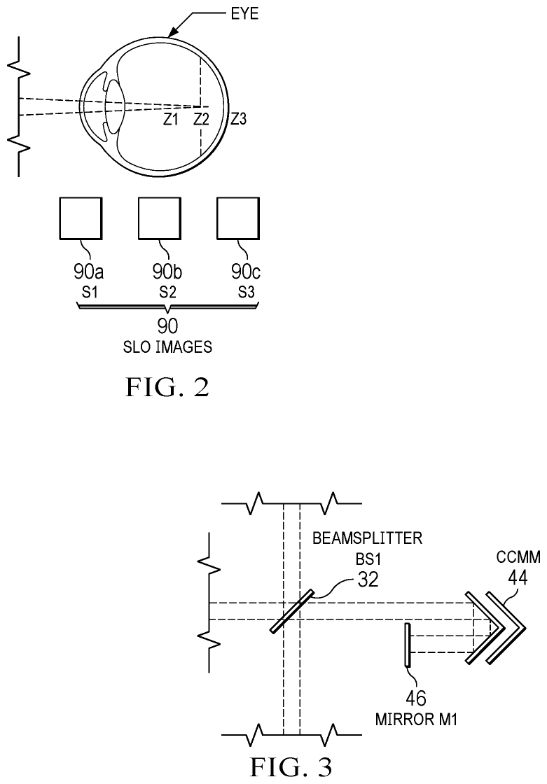

FIG. 2 illustrates examples of SLO 2D enface images 90 (90a, 90b, 90c) that may be generated by SLO device 24 of FIG. 1, according to certain embodiments. Different 2D enface images 90 image xy-planes at different z-locations. For example, image S1 90a images the xy-plane at z1, image S2 90b images the xy-plane at z2, and image S3 90c images the xy-plane at z3.

In certain embodiments, computer 23 generates a 3D image from the 2D images. Computer 23 aligns the 2D images in order according to their z-locations and then generates the 3D image from the aligned 2D images. For example, computer 23 corrects each image 90a, 90b, 90c for eye movement during the z-scan to yield a corrected 3D data set by laterally matching and shifting each image with respect to the previous image within the z-scan. Computer 23 may perform further processing and analysis of the corrected 3D data set. For example, computer 23 may calculate a topography and/or a reflectance image from the 3D data set. As another example, computer 23 may analyze the z-profile for each xy-pixel to detect floater in the corresponding 2D image.

FIG. 3 illustrates an example of corner cube moving mirror CCMM 44 and mirror M1 46 of z-scanner 38 of FIG. 1, according to certain embodiments. CCMM 44 is used to adjust the path length to create a coherence gate where paths are phase matched for maximum signal. CCMM 44 moves relative to beamsplitter BS1 32 and mirror M1 46 to match the optical path length of the OCT/SLO imaging path and laser beam path in the z-direction. For example, CCMM 44 moves to change the length of the reference arm of OCT device 26, the focus of the SLO imaging beam, and/or the focus of the laser beam. CCMM 44 allows for greater alignment tolerance, as CCMM 44 does not have to be perfectly aligned to direct beams back to beamsplitter BS1 32.

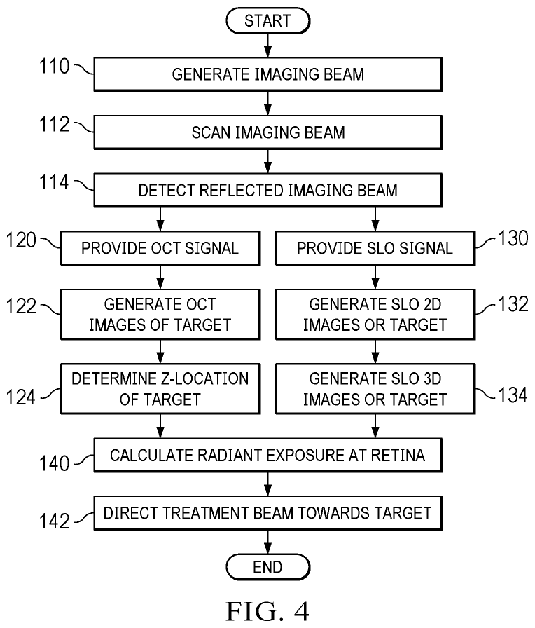

FIG. 4 illustrates an example of a method for imaging and fragmenting a target in an eye, which may be performed by system 10 of FIG. 1, according to certain embodiments. The method starts at step 110, where an imaging laser source generates an imaging beam. A scanning system scans the imaging beam within the eye at step 112. For example, the scanning system includes an xy-scanner that scans beams in the xy-direction and a z-scanner that scans beams in the z-direction. A computer may instruct an OCT device to scan an OCT imaging beam in the z-direction to generate an A-scan and instruct an SLO device to scan an SLO imaging beam in the xy-direction to generate two-dimensional (2D) enface images. The OCT and SLO imaging beams may scanned simultaneously.

The eye reflects the imaging beam. A detector system detects the reflected imaging beam at step 114. The detector system provides an OCT signal to an OCT detector at step 120. For example, a LF filter provides the OCT signal to the OCT detector. The OCT device generates OCT images of target at step 122. The OCT device determines the z-location of target at step 124. The detector system provides an SLO signal to an SLO detector at step 130. For example, a HF filter provides an SLO signal to the SLO detector. The SLO device generates 2D SLO enface images of target at step 132. Each enface image may be located in a different xy-plane. The computer generates 3D images of the target at step 134 from the 2D images. The computer may combine aligned 2D enface images to yield a 3D SLO image of the target.

The computer calculates at step 140 the radiant exposure at the retina resulting from a laser beam directed to the z-location of the target. In certain embodiments, the computer calculates the radiant exposure Hₑ by determining a laser spot size of the laser beam on the retina, and calculating the radiant exposure Hₑ according to the target z-location and the laser spot size. In the embodiments, the computer may determine whether the radiant exposure is greater or less than a maximum radiant exposure. If the radiant exposure is greater than the maximum acceptable radiant exposure, the computer may notify the user, adjust the laser energy, and/or prevent the laser from firing. A treatment system directs the laser beam towards the target at step 142 to fragment and remove the target. The laser device may receive the xy- and z-locations of the target from the imaging system and direct the laser beam towards the xy- and z-locations. The method then ends.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic laser surgical system for imaging and treating a target in an eye, comprising:
an imaging system configured to direct a plurality of imaging beams into the eye to generate a plurality of images of the target within the eye, the plurality of imaging beams comprising a scanning laser ophthalmoscope (SLO) imaging beam and an optical coherence tomography (OCT) imaging beam, the eye having an eye axis, the eye axis defining a z-axis, the z-axis defining a plurality of xy-planes orthogonal to the z-axis, the imaging system comprising:
an SLO device comprising:
a scanning system configured to scan the SLO imaging beam within the eye;
a light detector configured to generate an SLO signal in response to detecting the SLO imaging beam reflected from the eye; and
an SLO detector configured to generate a plurality of SLO images from the SLO signal; and
an OCT device comprising:
the scanning system configured to scan the OCT imaging beam within the eye;
the light detector configured to generate an OCT signal in response to detecting the OCT imaging beam reflected from the eye; and
an OCT detector configured to generate a plurality of OCT images from the OCT signal; and
a treatment system comprising a laser device, the laser device configured to direct a laser beam towards the target within the eye; and
a computer configured to:
instruct the imaging system to generate the plurality of images; and
instruct the laser device to direct the laser beam towards the target.

2. The ophthalmic laser surgical system of claim 1, further comprising:
an imaging beam source configured to generate the plurality of imaging beams comprising the SLO imaging beam and the OCT imaging beam.

3. The ophthalmic laser surgical system of claim 1, the computer configured to:
instruct the OCT device to scan the OCT imaging beam in a z-direction relative to the z-axis to generate an A-scan within the eye; and
instruct the SLO device to scan the SLO imaging beam in an xy-direction relative to the xy-planes to generate a plurality of two-dimensional (2D) enface images.

4. The ophthalmic laser surgical system of claim 1, the scanning system comprising:
an xy-scanner configured to scan an imaging beam in an xy-direction relative to an xy-plane within the eye; and
a z-scanner configured to scan the imaging beam in a z-direction relative to the z-axis within the eye.

5. The ophthalmic laser surgical system of claim 4, the xy-scanner configured to:
direct the imaging beams along an imaging beam path towards an xy-location of the target; and
direct the laser beam along a laser beam path aligned with the imaging beam path towards the xy-location of the target.

6. The ophthalmic laser surgical system of claim 4, the z-scanner configured to:
direct the imaging beams along an imaging beam path towards a z-location of the target; and
direct the laser beam along a laser beam path aligned with the imaging beam path towards the z-location of the target.

7. The ophthalmic laser surgical system of claim 4, the z-scanner comprising:
a corner cube moving mirror (CCMM) configured to move to adjust a path length to create a coherence gate for maximum signal.

8. The ophthalmic laser surgical system of claim 1, the light detector comprising:
a high-frequency filter configured to provide the SLO signal; and
a low-frequency filter configured to provide the OCT signal.

9. The ophthalmic laser surgical system of claim 1, the OCT detector comprising:
a fringe counter configured to count interference fringes.

10. The ophthalmic laser surgical system of claim 1, the OCT device configured to:
measure a z-location of the target relative to the z-axis.

11. The ophthalmic laser surgical system of claim 1, the laser device configured to:
receive a z-location of the target from the imaging system; and
direct the laser beam towards the z-location of the target.

12. The ophthalmic laser surgical system of claim 1, the computer configured to:
determine a radiant exposure at a retina of the eye resulting from the laser beam directed to a z-location of the target; and
determine whether the radiant exposure is less than a maximum radiant exposure.

13. The ophthalmic laser surgical system of claim 1:
the SLO device configured to generate a plurality of two-dimensional (2D) enface images, each enface image located in a different xy-plane; and
the computer configured to combine the plurality of 2D enface images to yield one or more three-dimensional (3D) images.

14. The ophthalmic laser surgical system of claim 13, the computer further configured to output the 3D images via a display.

15. A method for imaging and treating a target in an eye, comprising:
directing, by an imaging system, a plurality of imaging beams into the eye to generate a plurality of images of the target within the eye, the plurality of imaging beams comprising a scanning laser ophthalmoscope (SLO) imaging beam and an optical coherence tomography (OCT) imaging beam, the eye having an eye axis, the eye axis defining a z-axis, the z-axis defining a plurality of xy-planes orthogonal to the z-axis, the generating the images of the target comprising:
scanning, by a scanning system of an SLO device of the imaging system, the SLO imaging beam within the eye;
generating, by a light detector of the SLO device, an SLO signal in response to detecting the SLO imaging beam reflected from the eye;
generating, by an SLO detector of the SLO device, a plurality of SLO images from the SLO signal;
scanning, by the scanning system of an OCT device of the imaging system, the OCT imaging beam within the eye;
generating, by the light detector of the OCT device, an OCT signal in response to detecting the OCT imaging beam reflected from the eye; and
generating, by an OCT detector of the OCT device, a plurality of OCT images from the OCT signal;
directing, by a laser device of a treatment system, a laser beam towards the target within the eye;
instructing, by a computer, the imaging system to generate the plurality of images; and
instructing, by the computer, the laser device to direct the laser beam towards the target.

16. The method of claim 15, further comprising:
instructing, by the computer, the OCT device to scan the OCT imaging beam in a z-direction relative to the z-axis to generate an A-scan within the eye; and
instructing, by the computer, the SLO device to scan the SLO imaging beam in an xy-direction relative to the xy-planes to generate a plurality of two-dimensional (2D) enface images.

17. The method of claim 15, further comprising:
scanning, by an xy-scanner of the scanning system, an imaging beam in an xy-direction relative to an xy-plane within the eye; and
scanning, by a z-scanner of the scanning system, the imaging beam in a z-direction relative to the z-axis within the eye.

18. The method of claim 17, further comprising:
directing, by the xy-scanner, the imaging beams along an imaging beam path towards an xy-location of the target; and
directing, by the xy-scanner, the laser beam along a laser beam path aligned with the imaging beam path towards the xy-location of the target.

19. The method of claim 17, further comprising:
directing, by the z-scanner, the imaging beams along an imaging beam path towards a z-location of the target; and
directing, by the z-scanner, the laser beam along a laser beam path aligned with the imaging beam path towards the z-location of the target.

20. The method of claim 17, the z-scanner comprising:
a corner cube moving mirror (CCMM) configured to move to adjust a path length to create a coherence gate for maximum signal.
